(19) United States
(12) Patent Application Publication
(10) Pub. No.: US 2012/0225956 A1
(43) Pub. Date: Sep. 6, 2012

(54) Catalysts For The Reduction Of Carbon Dioxide To Methanol

(75) Inventors: Felix Studt, San Francisco, CA (US); Frank Abild-Pedersen, Menlo Park, CA (US); Jens K. Norskov, Menlo Park, CA (US); Soren Dahl, Hillerod (DK); Irek Sharafutdinov, Vedbaek (DK); Christian F. Elkjaer, Kobenhaven (DE)

(73) Assignee: The Board of Trustees of the Leland Stanford Junior University, Palo Alto, CA (US)

(21) Appl. No.: 13/411,358

(22) Filed: Mar. 2, 2012

Related U.S. Application Data
(60) Provisional application No. 61/464,482, filed on Mar. 4, 2011.

(51) Int. Cl. C07C 29/156; B01J 23/62; B01J 21/02; B01J 23/60; C07C 29/157; B01J 23/80; B01J 23/825; B82Y 30/00

(52) U.S. Cl. 518/709; 502/329; 502/333; 502/335; 502/332; 518/715; 977/773; 977/810

(57) ABSTRACT

A catalytic composition is provided for methanol production. The composition includes an alloy of at least two different metals M and M′, where M is selected from Ni, Pd, Ir, and Ru, and M′ is selected from Ga, Zn, and Al. A molar ratio of M to M′ is in the range of 1:10 to 10:1, and the alloy is configured to catalyze a reduction of CO₂ to methanol.

### CROSS-REFERENCE TO RELATED APPLICATION

This application claims the benefit of U.S. Provisional Application Ser. No. 61/464,482 filed on Mar. 4, 2011, the disclosure of which is incorporated herein by reference in its entirety.

### STATEMENT REGARDING FEDERALLY SPONSORED RESEARCH OR DEVELOPMENT

This invention was made with Government support under Grant No. DE-AC02-76SF00515, awarded by the Department of Energy. The Government has certain rights in this invention.

### FIELD OF THE INVENTION

The invention generally relates to the production of methanol and, more particularly, to catalysts for the production of methanol.

### BACKGROUND

Nature reduces carbon dioxide (CO₂) photo-chemically to store energy, and it remains one of the grand challenges in modern chemistry to design a process and catalysts to do the same. An initial stage in such a process could involve the generation of molecular hydrogen through a photo-electrochemical process or an electrochemical process using electrical power from photovoltaic cells or wind turbines. If this initial stage is followed by a heterogeneously catalyzed process to reduce CO₂ to methanol, a sustainable source of a liquid fuel would have been established. Although certain copper-based (Cu-based) catalysts are currently used for industrial, hydrocarbon-based processes, these catalysts may not be appropriate for the reduction of CO₂ to methanol, particularly if such reduction is carried out in smaller scale, decentralized plants. Specifically, Cu-based catalysts can suffer from complex synthesis as well as deactivation that is substantially irreversible.

It is against this background that a need arose to develop the catalysts and related systems and processes described herein.

### SUMMARY

One aspect of the invention relates to a catalytic composition for methanol production. In one embodiment, the composition includes an alloy of at least two different metals M and M′, wherein M is selected from Ni, Pd, Ir, and Ru, and M′ is selected from Ga, Zn, and Al. A molar ratio of M to M′ is in the range of 1:10 to 10:1, and the alloy is configured to catalyze a reduction of CO₂ to methanol.

Another aspect of the invention relates to a process for methanol production. In one embodiment, the process includes: (a) providing a catalyst including at least two different metals M and M′, wherein M is selected from transition metals of Group 8, transition metals of Group 9, and transition metals of Group 10, and M′ is selected from transition metals of Group 4, transition metals of Group 12, and post-transition metals of Group 13; and (b) contacting a feed stream including CO₂ with the catalyst.

Other aspects and embodiments of the invention are also contemplated. The foregoing summary and the following detailed description are not meant to restrict the invention to any particular embodiment but are merely meant to describe some embodiments of the invention.

### BRIEF DESCRIPTION OF THE DRAWINGS

For a better understanding of the nature and objects of some embodiments of the invention, reference should be made to the following detailed description taken in conjunction with the accompanying drawings.

FIG. 1: A system for the production of methanol implemented in accordance with an embodiment of the invention.

FIGS. 2 and 3: Theoretical activity volcano for methanol synthesis. The turnover frequency (TOF) is plotted as a function of carbon and oxygen binding energies (ΔE_{C} and ΔE_{O}). ΔE_{C} and ΔE_{O} for the stepped 211 surfaces of selected transition metals are depicted in FIG. 2. ΔE_{C} and ΔE_{O} for binary alloys are depicted in FIG. 3.

FIG. 4: a) Activity towards methanol of a series of NiₐGa_{b} catalysts compared to Cu/ZnO/Al₂O₃ as a function of temperature at atmospheric pressure. Gas composition: 75% H₂ and 25% CO₂. Gas hourly space velocity (GHSV)=6000 s⁻¹. b) Selectivity towards methanol in % as a function of temperature.

FIG. 5: (top) Transmission electron microscopy (TEM) images of Ni₅Ga₃ and NiGa. (bottom) In-situ X-ray diffraction (XRD) spectra of Ni₃Ga, NiGa, Ni₅Ga₃ alloys.

FIG. 6: Deactivation and reactivation of Ni₅Ga₃ with time on stream.

FIG. 7: Activity of Ni₅Ga₃ and Cu/ZnO/Al₂O₃ catalysts at 1 bar and 5 bar.

FIG. 8: Activity of NiₐGa_{b} catalyst at different gas compositions.

FIG. 9: Comparison of methanol synthesis activity at 1 bar pressure and varying temperatures. About 0.47 g of about 17 wt. % of NiₐGa_{b} catalyst was tested against about 0.17 g of the as-prepared Cu/ZnO/Al₂O₃ catalyst. The Cu-based catalyst showed slightly higher activity at lower temperatures, whereas the NiₐGa_{b} catalyst has a higher methanol yield at higher temperatures due to a lower reverse water-gas-shift activity.

FIG. 10: Reduction at varying temperatures followed by methanol synthesis reaction at about 180° C. All three reduction temperatures produced methanol, but the yield is highest after 600° C. and 700° C. The black markers correspond to XRD spectra shown in FIG. 11.

FIG. 11: XRD spectra for NiₐGa_{b} catalyst after reduction at three different temperatures. After 500° C., the alloy phase is Ni₃Ga, whereas after 600° C. and 700° C., the spectra show a Ni₅Ga₃ phase.

### DETAILED DESCRIPTION

### Definitions

The following definitions apply to some of the aspects described with respect to some embodiments of the invention. These definitions may likewise be expanded upon herein.

As used herein, the singular terms “a,” “an,” and “the” include plural referents unless the context clearly dictates otherwise. Thus, for example, reference to an object can include multiple objects unless the context clearly dictates otherwise.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable characteristics that are substantially the same as those of the non-spherical object. Thus, for example, a size of a non-spherical object can refer to a diameter of a corresponding spherical object that exhibits light scattering characteristics that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

### Catalysts for Reduction of Carbon Dioxide

Embodiments of the invention are directed to improved catalysts for methanol synthesis, which are active and selective towards methanol as the main product. Some embodiments are designed based on a model that reduces the energy parameters that describe methanol synthesis to two: the carbon and oxygen adsorption energies. A computational search for materials with optimal values of these two parameters is then used to identify catalyst leads.

Based on such modeling, improved catalysts for methanol production can be provided in the form of metal compositions, including alloys, intermetallic compounds, mixtures, or other compositions including two or more different metals and optionally other elements, such as in the form of dopants. Some embodiments can be provided as metal alloys including at least two different metals M and M′, where M can be one or more of transition metals of Group 8 (e.g., ruthenium (Ru)), transition metals of Group 9 (e.g., rhodium (Rh) and iridium (Ir)), and transition metals of Group 10 (e.g., nickel (Ni), palladium (Pd), and platinum (Pt)), and M′ can be one or more of transition metals of Group 4 (e.g., hafnium (HO), transition metals of Group 12 (e.g., zinc (Zn)), and post-transition metals of Group 13 (e.g., aluminum (Al) and gallium (Ga)). More particularly, M can be one or more of Ni, Pd, Ir, and Ru, and M′ can be one or more of Ga, Zn, and Al. Even more particularly, M can be Ni, and M′ can be Ga or Zn.

In some embodiments, a catalyst includes a binary metal alloy that can be represented as MₐM′_{b}, where a molar ratio of M to M′ can be represented as M:M′ corresponding to a:b (or a/b), which, in some embodiments, can be in the range of about 1:20 (or about 1/20) to about 20:1 (or about 20/1), such as from about 1:15 (or about 1/15) to about 15:1 (or about 15/1) or from about 1:10 (or about 1/10) to about 10:1 (or about 10/1). More particularly, the molar ratio of M to M′ can be greater than or equal to about 1:1 (or about 1/1), such as at least about 1:1 (or about 1/1) and up to about 20:1 (or about 20/1), such as up to about 15:1 (or about 15/1), up to about 10:1 (or about 10/1), up to about 5:1 (or about 5/1), up to about 4:1 (or about 4/1), up to about 3:1 (or about 3/1), up to about 2:1 (or about 2/1), or up to about 5:3 (or about 5/3). Even more particularly, the molar ratio of M to M′ can be greater than about 1:1 (or about 1/1), such as at least about 1.5:1 (or about 1.5/1) and up to about 20:1 (or about 20/1), such as up to about 15:1 (or about 15/1), up to about 10:1 (or about 10/1), up to about 5:1 (or about 5/1), up to about 4:1 (or about 4/1), up to about 3:1 (or about 3/1), up to about 2:1 (or about 2/1), or up to about 5:3 (or about 5/3). Examples of binary metal alloys useful as catalysts for methanol production include those represented as NiₐGa_{b}, such as Ni₅Ga₃, Ni₃Ga, and NiGa. Additional examples of binary metal alloys useful as catalysts include those represented as NiₐZn_{b}, such as Ni₅Zn₃, Ni₃Zn, and NiZn, and those represented as PdₐGa_{b}, such as Pd₅Ga₃, Pd₃Ga, and PdGa. Other embodiments can be provided as ternary, quaternary, or higher order metal alloys including three or more different metals and optionally other elements, such as in the form of dopants. In some embodiments, such ternary or higher order metal alloys can include the metals M and M′ having the characteristics and molar ratios as set forth above, in which at least one of M and M′ includes two or more different metals.

A catalyst of some embodiments can be provided in a particulate form, such as in the form of particles having an average size (e.g., an average size in a number or count distribution) in the range of about 1 nm to about 200 nm, such as from about 1 nm to about 100 nm, from about 1 nm to about 50 nm, from about 1 nm to about 20 nm, from about 1 nm to about 15 nm, or from about 1 nm to about 10 nm. For some embodiments, a catalyst has a surface area in the range of about 1 m²/g to about 500 m²/g (or greater), such as from about 10 m²/g to about 500 m²/g, from about 50 m²/g to about 500 m²/g, from about 50 m²/g to about 300 m²/g, from about 50 m²/g to about 200 m²/g, or from about 50 m²/g to about 100 m²/g. Such particle size and surface area can enhance exposure of a feed stream to active sites for improved catalytic activity.

In some embodiments, a catalyst support can be combined with a catalyst to provide mechanical support for the catalyst as well as to further enhance exposure of a feed stream to active sites of the catalyst. In such a supported configuration, an amount of the catalyst (represented as a weight loading of the catalyst relative to a total weight) can be in the range of about 0.1 wt. % to about 80 wt. %, such as from about 1 wt. % to about 70 wt. %, from about 5 wt. % to about 70 wt. %, from about 10 wt. % to about 70 wt. %, from about 10 wt. % to about 60 wt. %, from about 10 wt. % to about 50 wt. %, from about 10 wt. % to about 40 wt. %, from about 10 wt. % to about 30 wt. %, or from about 10 wt. % to about 20 wt. %. Examples of suitable catalyst supports include those based on silica (SiO₂), alumina (Al₂O₃), zirconia (ZrO₂), titania (TiO₂), MgAl₂O₃, and combinations thereof. A catalyst support can be porous or non-porous, and, in some embodiments, a catalyst support can be provided in a particulate form, such as in the form of particles having a surface area in the range of about 100 m²/g to about 400 m²/g, such as from about 200 m²/g to about 300 m²/g, a pore volume in the range of about 0.1 cm³/g to about 10 cm³/g, such as from about 0.5 cm³/g to about 5 cm³/g, and a median pore diameter in the range of about 1 nm to about 50 nm, such as from about 5 nm to about 30 nm.

A catalyst can be combined with a catalyst support or other support medium through, for example, impregnation or co-precipitation, such that the catalyst can be coated on, deposited on, impregnated on, incorporated into, or otherwise disposed adjacent to the catalyst support. For example, a supported catalyst can be synthesized through incipient wetness impregnation of an aqueous, pre-catalyst solution of a source of M (e.g., a salt of M) and a source of M′ (e.g., a salt of M′) on a catalyst support at a temperature in the range of about 20° C. to about 100° C. or about 20° C. to about 25° C., followed by exposure to molecular hydrogen (H₂) at an elevated temperature in the range of about 200° C. to about 1000° C., such as from about 200° C. to about 800° C. or from about 600° C. to about 800° C., and for a time period in the range of about 0.5 hour (h) to about 10 h, such as from about 0.5 h to about 5 h or from about 0.5 h to about 3 h. Advantageously, such synthesis can be readily carried out in an inexpensive and scalable manner, while avoiding complex synthesis of other types of catalysts.

The catalysts described herein can exhibit a high activity and a high selectivity for the production of methanol from a feed stream including CO₂. In some embodiments, the catalysts can exhibit an activity that is at least about 0.025 mole of methanol/[(mole of catalyst)·h], such as at least about 0.05 mole of methanol/[(mole of catalyst)·h], at least about 0.1 mole of methanol/[(mole of catalyst)·h], at least about 0.15 mole of methanol/[(mole of catalyst)·h], or at least about 0.2 mole of methanol/[(mole of the catalyst)·h], and up to about 0.8 mole of methanol/[(mole of catalyst)·h] (or greater), such as up to about 0.6 mole of methanol/[(mole of catalyst)·h], up to about 0.5 mole of methanol/[(mole of catalyst)·h], up to about 0.4 mole of methanol/[(mole of catalyst)·h], or up to about 0.3 mole of methanol/[(mole of catalyst)·h], when measured at a temperature of about 200° C. and a pressure of about 1 bar. And, in some embodiments, the catalysts can exhibit a selectivity towards the production of methanol (relative to other products or by-products) that is at least about 50%, such as at least about 60%, at least about 70%, at least about 80%, at least about 90%, or at least about 95%, and up to about 99.99%, such as up to about 99.9%, up to about 99.8%, up to about 99.5%, or up to about 99%, when measured at a temperature of about 200° C. and a pressure of about 1 bar, and when expressed as a percentage of methanol relative to a total amount of products in terms of moles, weight, or volume.

Furthermore, the catalysts described herein can exhibit other desirable characteristics. For example, the catalysts can be readily reactivated by reduction, such as by exposure to H₂ (e.g., substantially pure H₂) at an elevated temperature in the range of about 200 CC to about 800° C., such as from about 200° C. to about 400° C., and for a time period in the range of about 0.5 h to about 10 h, such as from about 1 h to about 7 h. Also, the catalysts can exhibit improved thermal stability (e.g., relative to Cu-based catalysts) by having a greater immunity against sintering at elevated temperatures. Also, the catalysts can be characterized by a low reverse water-gas-shift activity compared to other types of catalysts, which is favorable when a feed stream including a high proportion of CO, is used for methanol production. One advantage of a low reverse water-gas-shift activity can be a higher equilibrium methanol concentration and a reduced amount of water in the methanol product, thereby avoiding or simplifying downstream operations for removal of water.

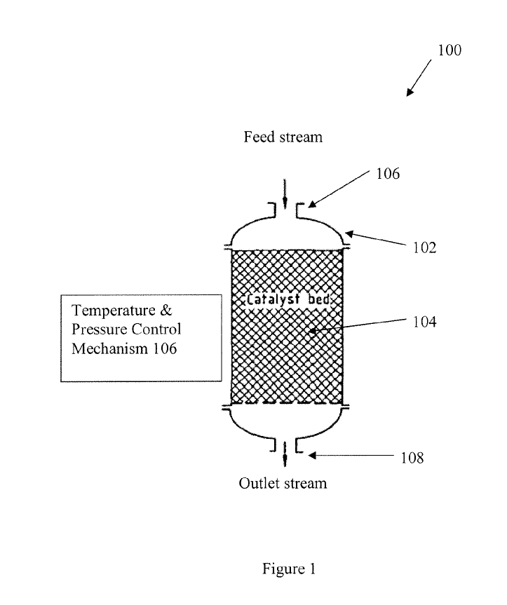

Attention next turns to FIG. 1, which illustrates a system 100 for the production of methanol according to an embodiment of the invention. The system 100 includes a catalytic reactor 102, which, in the illustrated embodiment, is implemented as a fixed-bed reactor, although other types of reactors are also contemplated. As illustrated in FIG. 1, the reactor 102 includes an inlet 106 through which a feed stream enters the reactor 102, and an outlet 108 through which an outlet stream exists the reactor 102.

The feed stream can include CO₂, H₂, and optionally another one or more gaseous components, such as carbon monoxide (CO), an inert gas (e.g., argon (Ar)), or a combination thereof. In some embodiments, the feed stream includes CO₂ and H₂ as the predominant components, such as collectively amounting to greater than 50%, such as at least about 60%, at least about 70%, at least about 80%, or at least about 90%, and up to about 100%, such as up to about 98% or up to about 95%, when expressed as a percentage of CO₂ and H₂ relative to a total amount of components in the feed stream in terms of moles, weight, or volume. A ratio of CO, to H₂ can be in the range of about 1:20 (or about 1/20) to about 20:1 (or about 20/1), such as from about 1:15 (or about 1/15) to about 15:1 (or about 15/1), from about 1:10 (or about 1/10) to about 10:1 (or about 10/1), or from about 1:5 (or about 1/5) to about 5:1 (or about 5/1), when expressed in terms of moles, weight, or volume. For example, the ratio of CO₂ to H₂ can be greater than or equal to about 1:1 (or about 1/1), such as at least about 1:1 (or about 1/1) and up to about 20:1 (or about 20/1), such as up to about 15:1 (or about 15/1), up to about 10:1 (or about 10/1), up to about 5:1 (or about 5/1), up to about 4:1 (or about 4/1), up to about 3:1 (or about 3/1), or up to about 2:1 (or about 2/1), when expressed in terms of moles, weight, or volume. In some embodiments, CO can be included in the feed stream (if at all) as a minority component, such as amounting to less than 50%, such as no greater than about 40%, no greater than about 30%, no greater than about 20%, no greater than about 10%, no greater than about 5%, no greater than about 2%, or greater than about 1%, when expressed as a percentage of CO relative to a total amount of components in the feed stream in terms of moles, weight, or volume.

Within the reactor 102, reduction of the feed stream takes place in the form of a heterogeneously catalyzed gas reaction on the surface of a catalyst (or a combination of different catalysts) as described herein, which, in the illustrated embodiment, is implemented in a supported configuration as a catalytic bed 104. Specifically, the feed stream is exposed to, or contact with, the catalytic bed 104, and converted into methanol that is included in the outlet stream.

As illustrated in FIG. 1, the system 100 also includes a temperature and pressure control mechanism 106, which is coupled to the reactor 102 and operates to adjust or maintain reaction conditions at desired levels or ranges. The control mechanism 106 can be incorporated upstream or downstream of the reactor 102, or can be integrated as part of the reactor 102, depending on the particular implementation. In some embodiments, a reaction temperature can be in the range of about 100° C. to about 400° C., such as from about 100° C. to about 300° C. or from about 150° C. to about 250° C., and a reaction pressure can be in the range of about 0.5 bar to about 10 bar, such as from about 0.5 bar to about 5 bar or from about 0.5 bar to about 2 bar.

In some embodiments, the system 100 can have at least two operation modes, including a reaction mode in which the feed stream has a composition as set forth above, and a reactivation mode in which the feed stream has a different composition to allow reactivation of the catalyst included in the catalytic bed 104. For example and as described above, reactivation can be carried out by exposure to H₂ at an elevated temperature in the range of about 200° C. to about 800° C., such as from about 200° C. to about 400° C., and for a time period in the range of about 0.5 h to about 10 h, such as from about 1 h to about 7 h. It is contemplated that a separate inlet can be included in the reactor 102 through which a reactivation stream of H₂ can enter the reactor 102.

### EXAMPLES

The following examples describe specific aspects of some embodiments of the invention to illustrate and provide a description for those of ordinary skill in the art. The examples should not be construed as limiting the invention, as the examples merely provide specific methodology useful in understanding and practicing some embodiments of the invention.

### Example 1

This example sets forth the design, synthesis, and testing of improved alloy catalysts for methanol synthesis. A series of leads for alloy catalysts have been established on the basis of a computational descriptor-based approach. An active candidate including Ni and Ga, hereinafter designated as NiₐGa_{b}, was then synthesized, and catalytic testing shows high performance that is at least comparable to that of a Cu/ZnO/Al₂O₃ catalyst. The NiₐGa_{b} catalyst is characterized using electron microscopy and X-ray diffraction, and results indicate that NiₐGa_{b} particles of the catalyst predominantly include a single intermetallic compound.

A theoretical analysis was based on various reaction steps involved in methanol synthesis. A mean-field kinetic model of this scheme was developed. Deviations from a mean-field model can sometimes be observed in cases of strong adsorbate-adsorbate interactions or if surface diffusion is not sufficiently fast to allow equilibration on the surface at reaction conditions. For the relatively noble catalysts (like Cu) and other catalysts expected to be optimal for methanol synthesis, surface coverages are typically small, and diffusion is typically fast for all intermediates under reaction conditions.

Each elementary reaction step has a rate, rᵢ=νᵢ exp(−E_{u,i}/kT), which is calculated in harmonic transition state theory. The prefactors are calculated for each reaction step on catalyst (Cu) and used throughout. The activation energies for the forward elementary steps, together with the elementary reaction energies, have been calculated with Density Functional Theory (DFT) using the revised Perdew-Burke-Ernzerhof (RPBE) exchange-correlation energy functional for a selected set of metals. In each case, a stepped fcc(211) surface was selected to represent the active site.

The following describes an approach to reduce the number of energy parameters in the methanol synthesis to 2. In doing so, some accuracy may be lost, but it is desirable to develop such a model for at least two reasons. First, the model facilitates understanding of the trends in catalytic activity among the metals, and, second, the model provides a tractable way to search for new leads among numerous possible alloy catalysts. It has been found that scaling relations exist between the C and O adsorption energies, ΔE_{C} and ΔE_{O}, and the adsorption energies of hydrogenated forms of these atoms when different metals are compared. Generalizing the scaling relation concept to two-dimensions, it is determined that all reaction energies in the methanol synthesis scale with a combination of ΔE_{C} and ΔE_{O}. Similar scaling relations can be invoked for transition state energies (Brønsted-Evans-Polanyi relations). The result is a complete mapping of all the relevant energies in the methanol synthesis onto two parameters, ΔE_{C} and ΔE_{O}. To a first approximation, these parameters characterize the catalytic properties uniquely.

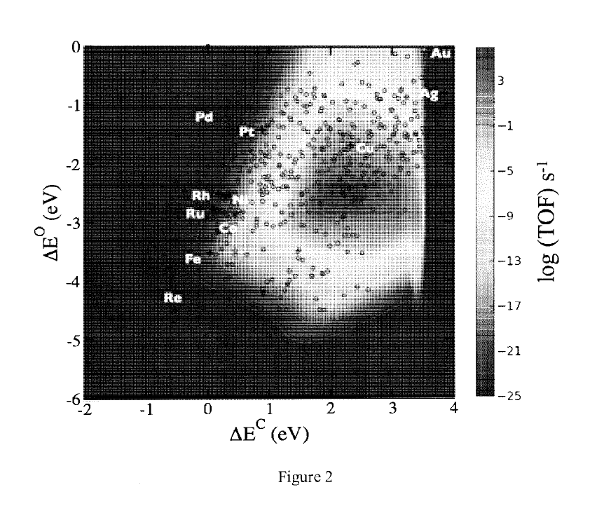

The calculated rate of methanol synthesis as a function of ΔE_{C} and ΔE_{O} is shown in FIG. 2. Values of (ΔE_{C}, ΔE_{O}) for a number of elemental transition metals are included in this two-dimensional volcano plot. The optimum in reaction rate is a result of a competition between having a weak interaction with C and O (resulting in unstable intermediates and high reaction barriers) and having a strong coupling to C (giving rise to a blocking of the surface with carbon bound species, such as CO) and O (giving rise to surface poisoning by formate, methoxy, OH, and other species bound through oxygen). The calculations show why Cu is one catalyst material of choice. It is noted that, while Cu is closest to the top, Cu is still not quite optimal (even when a typical error in the DFT calculations and in the scaling relations of 0.2 eV is taken into account). It has been suggested that one effect of adding Zn to the Cu catalyst is to promote the catalyst surface. The calculations show that adding Zn to the step of Cu(211) decreases ΔE_{O} by 0.33 eV (stronger bond), thereby moving it closer to the optimum. The mapping of the kinetics onto two descriptors thus provides an improved understanding of methanol synthesis catalysts.

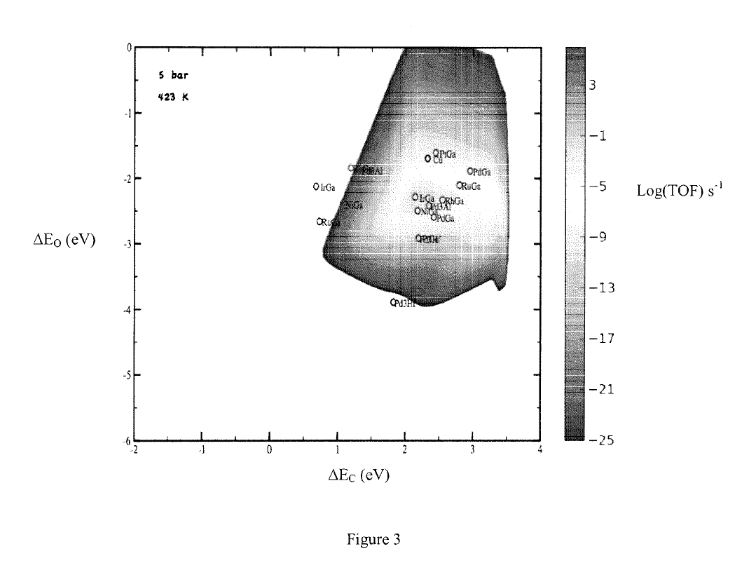

The two-descriptor model provides an efficient way to identify leads for improved catalysts. The model shows that the optimum catalyst is one that binds O stronger than Cu, while the C adsorption should be about the same. Thus, candidates were identified by calculating ΔE_{C} and ΔE_{O} for a range of alloys. FIG. 3 identifies alloy surfaces, as defined by (ΔE_{C}, ΔE_{O}), closest to optimum. Of those, the intermetallic compound NiGa stands out as being very stable. The heat of formation of NiGa is calculated to be about −1.27 eV/formula unit (4 atoms), resulting in a cohesive energy E_{coh} of about 4.26: this is considerably more stable than Cu (E_{coh}=3.49). NiGa is, therefore, expected to be less susceptible to sintering than Cu, and may not undergo the rapid deactivation that is observed for certain Cu-based catalysts.

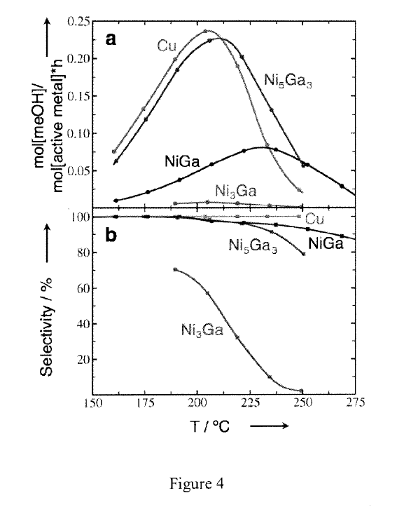

Having identified NiGa as a lead that is promising both with respect to activity and stability, a series of NiₐGa_{b} catalysts with different Ni to Ga ratios supported on silica were synthesized using incipient wetness impregnation. For comparison, a Cu/ZnO/Al₂O₃ catalyst was also synthesised. The NiₐGa_{b}/SiO₂ catalysts were tested for CO₂ hydrogenation at pressures of 1 bar in a tubular fixed-bed reactor. FIG. 4 shows the activity and selectivity towards methanol synthesis as a function of temperature for a series of NiₐGa_{b}/SiO₂ catalysts as well as Cu/ZnO/Al₂O₃. Among the NiₐGa_{b}/SiO₂ catalysts synthesized in this study, Ni₅Ga₃/SiO₂ stands out as being particularly active towards methanol, with an activity that is comparable to that obtained for Cu/ZnO/Al₂O₃. Selectivity towards methanol is quite high up to temperatures of about 200° C. and decreases slightly for higher temperatures. This decrease in selectivity may result from Ni particles that have not been alloyed with Ga, and hence produce methane in a side reaction. The other two NiₐGa_{b} catalysts tested, NiGa/SiO₂ and Ni₃Ga/SiO₂, are both less active, while NiGa/SiO₂ is slightly more selective then Ni₅Ga₃/SiO₂. Further optimizations in composition or synthesis can produce further enhancements in performance.

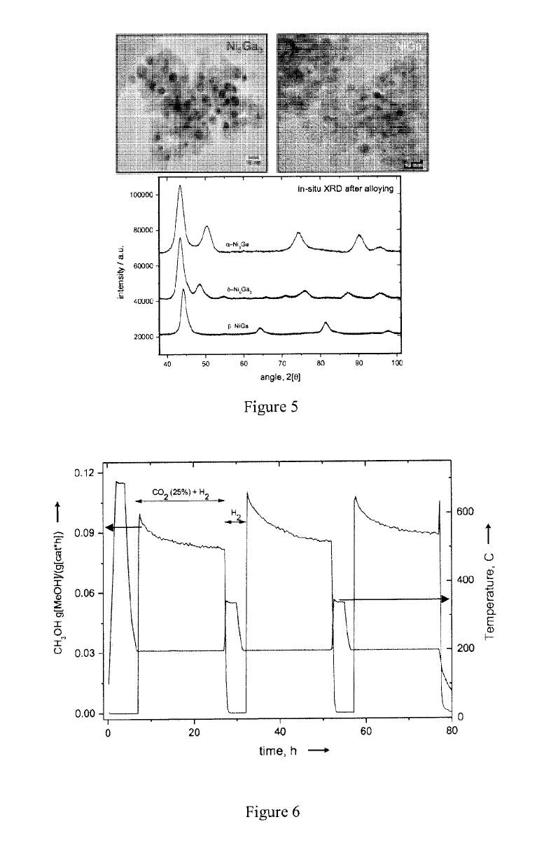

X-ray diffraction (XRD) spectra of the series of NiₐGa_{b} catalysts are shown in FIG. 5 together with transmission electron microscopy (TEM) images of Ni₅Ga₃ and NiGa particles. As can be seen from the XRD spectra, all three different NiₐGa_{b} alloys, Ni₃Ga, NiGa, and Ni₅Ga₃, can be synthesized as substantially phase pure. This purity can be attributed to the high formation energy of the different phases, and the sharp lines in the NiₐGa_{b} phase diagram. The TEM images shown in FIG. 5 reveal a size distribution with an average size of about 5.1 nm for Ni₅Ga₃ particles and about 6.2 nm for NiGa particles. Based on this size distribution, the active surface area per gram of catalyst can be estimated to be at least comparable to the Cu/ZnO/Al₂O₃ catalyst, where the combined surface area of Cu/ZnO/Al₂O₃ is about 92 m²/g.

Stability, which is an issue of the Cu/ZnO/Al₂O₃ catalyst, has been tested for Ni₅Ga₃/SiO₂. FIG. 6 shows the activity of Ni₅Ga₃/SiO₂ as a function of time on stream at about 200° C. and about atmospheric pressure. As can be seen from FIG. 6, Ni₅Ga₃/SiO₂ deactivates with time on stream, retaining about 80% of its initial activity after 20 hours on stream. Tests were conducted to reactivate Ni₅Ga₃ through reduction with hydrogen at about 350° C. for about 2 hours. As shown in FIG. 6, the Ni₅Ga₃ catalyst was substantially reactivated to its original activity after reduction. Reduction with hydrogen yields primarily methane as detected by gas chromatography, and hence it is expected that deactivation of Ni₅Ga₃ occurs primarily via carburization. Reactivation of the Cu/ZnO/Al₂O₃ catalyst was on the other hand not very successful, since sintering is typically the main cause for deactivation rather than carburization.

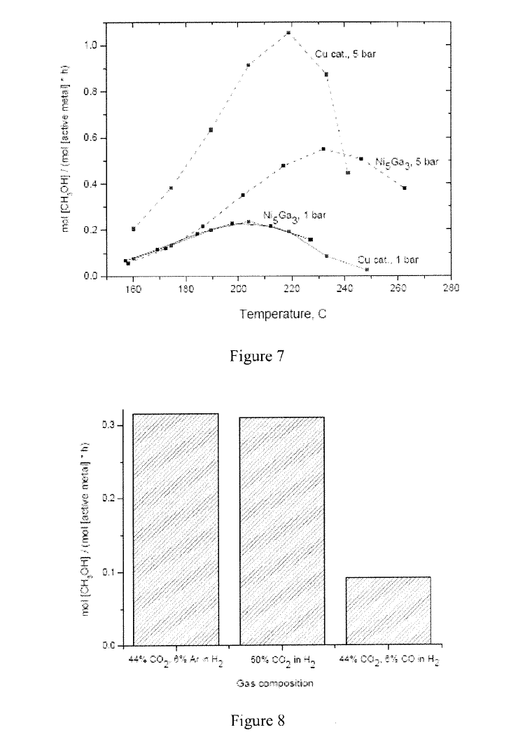

FIG. 7 shows the activity of the Cu/ZnO/Al₂O₃ and the Ni₅Ga₃/SiO₂ catalysts at 1 bar and 5 bar. An almost three fold increase in methanol yield is observed for the Cu/ZnO/Al₂O₃ catalyst when the pressure is increased from 1 bar to 5 bar, while a more modest increase is observed for the Ni₅Ga₃/SiO₂ catalyst. The results presented so far were obtained using CO₂ and H₂ as a reaction mixture, but the effect of adding CO was investigated as well. Generally, CO was observed to have a detrimental effect on the activity of the tested NiₐGa_{b} catalysts, as depicted in FIG. 8, which shows the yield of methanol at three different gas compositions.

In summary, the complex reaction scheme of methanol synthesis can be described through scaling and transition-state scaling relations to reduce the number of parameters to two. This simplification allowed for the screening of a number of binary alloys that can be potential leads for new methanol synthesis catalysts. Based on this screening procedure, binary NiₐGa_{b} alloys have been identified and synthesized. The performance of a series of NiₐGa_{b} alloys has been tested experimentally, and Ni₅Ga₃/SiO₂ was identified as a particularly active methanol catalyst. Of note, the activity of Ni₅Ga₃/SiO₂ at atmospheric pressures was at least comparable to Cu/ZnO/Al₂O₃. Although the Ni₅Ga₃/SiO₂ catalyst can deactivate due to carburization, substantially full reactivation can be readily achieved through reduction in hydrogen.

Experimental Section: DFT calculations for the intermediates and transition states were carried out on the (211) surfaces using the Dacapo code, which is available as open source software at http://wiki.fysik.dtu.dk/dacapo.

NiₐGa_{b} catalysts were synthesized using incipient wetness impregnation of a mixed aqueous solution of nickel and gallium nitrates (Sigma Aldrich) on silica (Saint-Gobain Norpo) at room temperature and at a constant pH of about 7. The samples were directly reduced at about 700° C. for about 2 h in hydrogen.

Activity measurements were carried out at a total flowrate of 100 Nml/min in a tubular fixed-bed reactor with a CO₂ to H₂ ratio of 3:1 at atmospheric pressures. The outlet stream was sampled every 15 min using a gas chromatograph (Agilent 7890A).

TEM measurements were performed using a FEI Technai TEM operating at 200 kV. XRD spectra were recorded with a PAN analytical X′Pert PRO diffractometer, which was equipped with an Anton Paar XRK in situ cell and a gas flow control system.

### Example 2

### Synthesis

A 17 wt. % NiₐGa_{b} catalyst supported on silica was prepared. The silica was high surface area silica from Saint-Gobain Norpro (SS 61138) with a surface area of about 257 m²/g, a pore volume of about 1 cm³/g, and a median pore diameter of about 11.1 nm. Nitride salts of Ni and Ga were dissolved in an amount of water corresponding to the pore volume of the silica support in the ratio Ni:Ga of about 64:36. The silica was then impregnated with this solution, followed by drying and aging at about 90° C. The catalyst was reduced inside a quartz reactor at about 700° C. and thereafter brought to reaction conditions for test of catalytic activity.

Catalytic Testing:

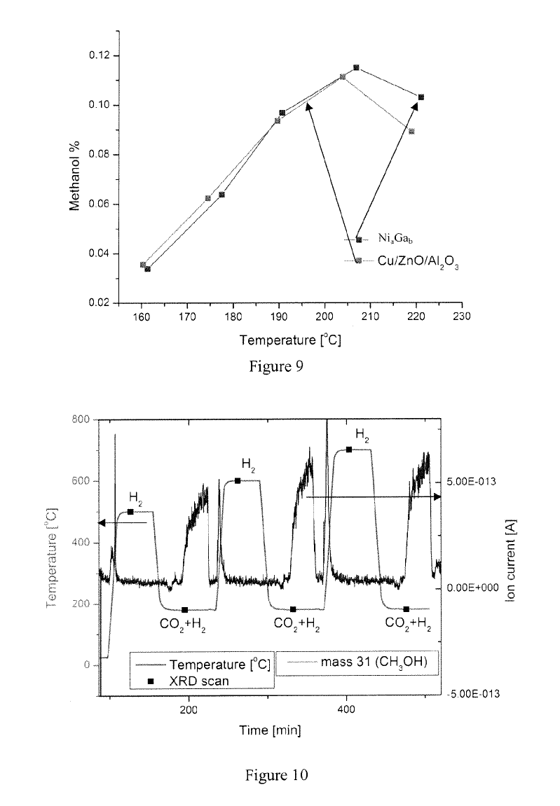

The catalyst activity was tested inside the same quartz reactor, and the NiₐGa_{b} catalyst was exposed to a gas mixture of about 25% CO₂ and about 75% H₂. The outflow was analyzed by gas chromatography, where a calibration was performed with known quantities of reactants and possible products including methanol. For the catalytic tests, the temperature in the reactor was varied by controlling an oven, and the pressure in the reactor could be varied by a pressure controller situated after the reactor. Results of such a test performed at about 1 bar under varying temperature is shown in FIG. 9.

In-Situ XRD:

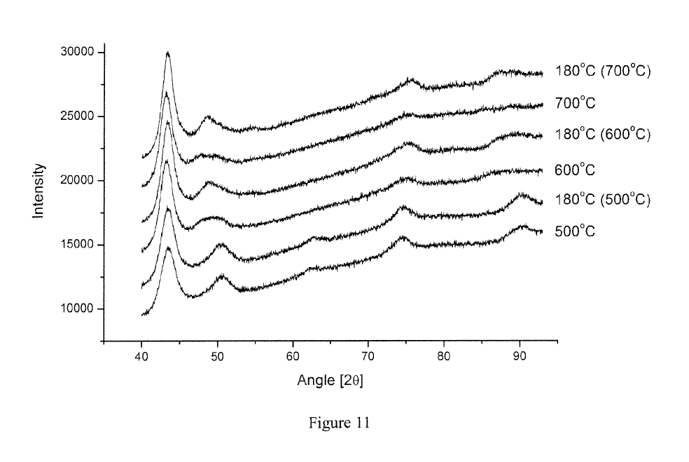

To determine the crystal phase of the NiₐGa_{b} catalyst, XRD was performed under controlled temperature and atmosphere, where the conditions mimicked those described above for synthesis and testing. Cu Kα X-rays were used. The prepared catalyst was reduced in pure hydrogen at an elevated temperature, and was then cooled to about 180° C. and exposed to a mixture of CO₂ and H₂ for testing of catalytic activity. This testing was carried out for reduction in three stages at about 500° C., about 600° C., and about 700° C. The outflow was monitored by mass spectrometry. The result of the catalytic testing is shown in FIG. 10, where a methanol yield was observed after all three stages, but was lowest after the 500° C. reduction. This observation can be related to the XRD spectra shown in FIG. 11, where a distinct Ni₃Ga phase is observed after reduction at 500° C., whereas after 600° C. and 700° C. the crystal changes to Ni₅Ga₃. Scherrer broadening of the largest peak at 43° indicates a particle size of about 5.5 nm after reduction at about 700° C.

Cu/ZnO/Al₂O₃ Catalyst for Comparison:

A Cu/ZnO/Al₂O₃ catalyst was prepared by co-precipitation. Specifically, about 60% Cu, about 30% Zn, and about 10% Al were precipitated by NaCO₃ at a constant pH of about 7, followed by 1 hour aging at a pH of about 7. Afterwards, a resulting gel was washed, dried, and calcined at about 300° C. Finally, the catalyst was reduced at about 200° C. under a flow of about 0.5% H₂ in Ar for about 20 hours. In-situ XRD was performed under similar conditions as described above, and yielded a particle size of about 5.5 nm after reduction, comparable to the particle size of the NiₐGa_{b} catalyst.

Results of Catalytic Tests:

About 0.47 g of the NiₐGa_{b} catalyst (corresponding to about 0.1 g of active metal) was tested against about 0.17 g (as weighed after calcination) of the Cu/ZnO/Al₂O₃ catalyst (corresponding to about 0.08 g of Cu). The results are shown in FIG. 9. As depicted in FIG. 9, the Cu/ZnO/Al₂O₃ catalyst was observed to be slightly more active than the NiₐGa_{b} catalyst at lower temperatures, although the NiₐGa_{b} catalyst provided a higher methanol yield at higher temperatures. This observation may result from the lower reverse water-gas-shift activity of the NiₐGa_{b} catalyst relative to the Cu/ZnO/Al₂O₃ catalyst.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A catalytic composition for methanol production, comprising:
an alloy of at least two different metals M and M′,
wherein
M is selected from Ni, Pd, Ir, and Ru, and M′ is selected from Ga, Zn, and Al,
a molar ratio of M to M′ is in the range of 1:10 to 10:1, and
the alloy is configured to catalyze a reduction of CO₂ to methanol.

2. The catalytic composition of claim 1, further comprising a support medium, and the alloy is disposed adjacent to the support medium.

3. The catalytic composition of claim 1, wherein M is Ni, and M′ is Ga or Zn.

4. The catalytic composition of claim 1, wherein the molar ratio of M to M′ is at least 1:1.

5. The catalytic composition of claim 4, wherein the molar ratio of M to M′ is up to 5:1.

6. The catalytic composition of claim 1, wherein the alloy is provided as particles having an average size in the range of 1 nm to 50 nm.

7. The catalytic composition of claim 6, wherein the average size is in the range of 1 nm to 10 nm.

8. The catalytic composition of claim 1, wherein the alloy has an activity that is at least 0.05 mole of methanol/[(mole of alloy)·h], as measured at a temperature of 200° C. and a pressure of 1 bar.

9. The catalytic composition of claim 8, wherein the activity is at least 0.15 mole of methanol/[(mole of alloy)·h].

10. A process for methanol production, comprising:
providing a catalyst including at least two different metals M and M′, wherein M is selected from transition metals of Group 8, transition metals of Group 9, and transition metals of Group 10, and M′ is selected from transition metals of Group 4, transition metals of Group 12, and post-transition metals of Group 13; and
contacting a feed stream including CO₂ with the catalyst.

11. The process of claim 10, wherein the catalyst includes an alloy of M and M′.

12. The process of claim 10, wherein M is selected from Ni, Pd, Ir, and Ru.

13. The process of claim 10, wherein M′ is selected from Ga, Zn, and Al.

14. The process of claim 10, wherein M is Ni, and M′ is Ga or Zn.

15. The process of claim 10, wherein contacting the feed stream with the catalyst is carried out at a reaction temperature in the range of 100° C. to 400° C. and a reaction pressure in the range of 0.5 bar to 10 bar.

16. The process of claim 15, wherein the reaction temperature is in the range of 100° C. to 300° C., and the reaction pressure is in the range of 0.5 bar to 5 bar.

17. The process of claim 10, wherein the feed stream includes CO₂ and H₂ collectively amounting to greater than 50% of the feed stream, expressed in terms of moles.

18. The process of claim 17, wherein a molar ratio of CO₂ to H₂ is at least 1:1.

19. The process of claim 15, further comprising reactivating the catalyst by contacting a reactivation stream including H₂ with the catalyst.
